(19) United States
(12) Patent Application Publication
(10) Pub. No.: US 2006/0216341 A1
(43) Pub. Date: Sep. 28, 2006

(54) Compositions for treating drug resistance

(76) Inventors: Paul Tardi, Surrey (CA); Troy Harasym, North Vancouver (CA); Murray Webb, North Vancouver (CA); Clifford Shew, Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 10/551,401

(22) PCT Filed: Apr. 2, 2004
(86) PCT No.: PCT/CA04/00506

§ 371 Date: Mar. 30, 2006

Related U.S. Application Data
(60) Provisional application No. 60/460,255, filed on Apr. 2, 2003.

(51) Int. Cl. A61K 9/127

(52) U.S. Cl. 424/450; 977/907

(57) ABSTRACT

Delivery vehicle compositions having stably associated therewith at least one therapeutic agent and at least one drug resistance modulator are useful in achieving a biologic effect to a drug resistance target.

### CROSS-RELATED TO RELATED APPLICATIONS

This application claims benefits under 35 USC 119(e) of U.S. Ser. No. 60/460,255 filed 2 Apr. 2003, the contents of which are incorporated herein by reference.

### TECHNICAL FIELD

The invention relates to compositions and methods for delivery of a therapeutic agent and a drug resistance modulator to a drug resistant target. More particularly, the invention concerns delivery vehicle compositions of a size range between 50 and 300 nm which provide combinations of therapeutic agents and drug resistance modulators to a drug resistance target.

### BACKGROUND OF THE INVENTION

Drug resistance is a major obstacle to the effective treatment of cancer as well as bacterial and viral infections. In the treatment of cancer, many tumors are initially responsive to chemotherapeutic agents but ultimately develop resistance to chemotherapy (Gioccone et al., Eur J Cancer (1995):31A(Suppl 7): 15-17). In addition, some cancers may also be inherently resistant to chemotherapy. Numerous mechanisms are known to contribute to drug resistance in tumors, including overexpression of drug efflux pumps, increased activity of DNA repair mechanisms, altered drug target enzymes and overexpression of enzymes involved in drug detoxification and elimination. Since many chemotherapy approaches ultimately elicit anticancer effects via apoptosis, alterations in the level of apoptosis control provide yet another mechanism by which drug resistance may occur. In the case of anti-bacterial treatment, a principal mechanism for antibiotic resistance is due to the presence of resistance genes that are carried on plasmids. These plasmids are independently replicated within and passed between bacterial cells and species.

Various groups have combined conventional therapeutic agents with drug resistance modulators to sensitize a resistant target. The rationale behind this approach has been to block mechanisms that lead to drug resistance so that the effects of the therapeutic agents can be realized. For instance, blockage of programmed cell death by the apoptosis regulating protein, Bcl-2, has also been implicated in mechanisms leading to chemoresistance. Combinations of the Bcl-2 antisense oligodeoxynucleotide, G3139, and liposomal doxorubicin have been shown to be more efficacious than either treatment alone, or when the G3139 was combined with free doxorubicin (Lopes de Menezes, et al., Clin Cancer Res (2000) 6:2891-2902) in the MDA435/LCC6 cell line. Another approach to treat drug resistance in cancer involves inhibiting the well-characterized p-glycoprotein (P-gp) drug efflux pump expressed in many drug resistance cells. Considerable improvements in the therapeutic activity of anticancer agents in conjunction with the P-gp inhibitor, PSC833, have been demonstrated in solid tumor MDR models (Boesch, et al., Cancer Res (1991) 51:4226; Watanabe et al., Acta Oncol (1995) 34:235; and Krishna et al., Int. J. Cancer (2000) 85:131-141). However, PSC833 has been known to cause toxicity in patients due to significant alterations in pharmacokinetics and biodistribution properties when co-administered (Lum and Gosland, Hematol Oncol Clin North Am (1995) 9:319).

Despite vast improvements in our understanding of the mechanism of drug resistance, the ability to treat the disorder has been complicated by the fact that agents used in combination to combat drug resistance may not display favourable pharmacokinetics after systemic administration. There are significant challenges in controlling the pharmacology of two or more agents that must work in concert in order to treat drug resistance without inducing significant deleterious side effects. Moreover, the treatment of drug resistance has been complicated due to the fact that it is often a multifaceted phenomenon and numerous interacting pathways control its development. It is thus also clear that there is a need for a multifunctional approach to combat drug resistance in which multiple pathways and intracellular proteins are simultaneously targeted in order to make a significant impact into improving the clinical activity of therapeutic agents.

This invention overcomes difficulties previously encountered to control the pharmacology of multiple agents that must work in concert to treat drug resistant targets. The present invention recognizes that it is possible to deliver a combination of a drug resistance modulator and a therapeutic agent to combat multiple drug resistance by controlling the pharmacokinetics of the formulation in which they are administered. This is achieved by stably associating the agents in delivery vehicles. It has also been recognized that the delivery vehicles must exhibit enhanced blood stability in order for optimal therapeutic benefits to be achieved. Enhancing the blood stability of the carriers may be achieved by preparing delivery vehicles of a size range that prevents uptake by the reticuloendothelial system (RES) and/or mononuclear phagocytic system (MPS) and/or by the incorporation of suitable surface stabilizing components in the delivery vehicles. Furthermore, it is recognized that multiple mechanisms that occur within the same cell may lead to the development of drug resistance. The modulation of these independent mechanisms can be achieved by the delivery of two or more drug resistance modulators in combination. Once again, this is achieved by stably associating the modulators in delivery vehicles and by controlling the pharmacokinetics of the formulation in which they are administered.

Rahman et al., in Journal of the National Cancer Institute (1992) 84(24): 1909-1915 have shown that doxorubicin-containing liposomes with cardiolipin incorporated within the bilayer modulate drug resistance in HL-60 cells that express p-glycoprotein. It was reported that direct interaction of the liposomes with p-glycoprotein was responsible for overcoming the drug resistance. The liposomes were small unilamellar liposomes (SUVs) and such liposomes are known to exhibit compromised circulation lifetime after intravenous administration. Thus, the drug carrier systems described in these papers would be of limited therapeutic benefit due to their inability to remain in the circulation for a sufficient time to reach a tumor site.

Matsuo et al., Journal of Controlled Release (2001) 77:77-86, have recently reported that vincristine-containing liposomes modified with the MRK-16, non-humanized monoclonal antibody directed against the p-glycoprotein drug efflux pump, were able to sensitize resistant cells in vitro. The results show that the cytotoxicity of vincristine encapsulated in liposomes containing the antibody to K-562/ADM cells was higher than that of vincristine encapsulated in control liposomes containing IgG. Although these results appear promising for the treatment of MDR, the liposomes described in this paper are of a mean diameter of greater than 400 nm and carriers of such a large size would be unstable in the bloodstream and prone to uptake by the RES and/or MPS. As well, the modification of liposome surfaces with antibodies is known to lead to rapid uptake due to the rapid recognition by cells of the immune system (Shek, et al., Immunology (1983) 50(1): 101-6; Aragnol, et al., Proc Natl Acad Sci USA (1986) 83(8): 2699-703).

Wu et al., in WO98/50018 describe the use of microsphere delivery vehicles containing a chemotherapeutic agent and a chemosensitizer to overcome drug resistance. The microspheres contain a biodegradable polymer matrix with functional groups which associate with the chemotherapeutic agent and the chemosensitizer. Intratumoral injection of the microspheres into drug resistant murine tumor models was carried out and a delay in the growth of the tumors was observed. Although these results appear promising, microspheres are delivery vehicles that are of a size (generally between 40 to 200 μm) that is not amenable to systemic administration.

Soma et al., Biomaterials (2000) 21:1-7 reported the successful co-encapsulation of cyclosporin A (CyA) with doxorubicin in polyalkylcyanoacrylate nanoparticles for the treatment of multidrug resistance. The nanoparticles employed in these experiments were greater than 200 nm.

### DISCLOSURE OF THE INVENTION

The invention relates to methods for administering a drug resistance modulator and a therapeutic agent or two drug resistance modulators using delivery vehicle compositions that are of a size range between 50 and 300 nm. Encapsulation in delivery vehicles allows the drug resistance modulator and the therapeutic agent to be delivered to a disease site in a coordinated fashion. The pharmacokinetics (PK) of the composition are controlled by the delivery vehicles themselves, such that coordinated delivery is achieved (provided that the PK of the delivery systems are comparable). By administering delivery vehicles that are in a size range of 50-300 nm, the associated agents are delivered to a target site such that a desired therapeutic effect is attained. This may be due to reduced uptake by the reticuloendothelial system (RES) and the mononuclear phagocyte system (MPS) and enhanced stability in the bloodstream. This result can be achieved whether the agents are co-encapsulated in delivery vehicles, or are separately encapsulated in delivery vehicles.

Thus, in one aspect, the invention provides a delivery vehicle composition for parenteral administration comprising a drug resistance modulator and a therapeutic agent. The delivery vehicles are of a size between 50 and 300 nm that allows for reduced clearance from the blood compartment. Stability in vivo may also be achieved by the incorporation of stabilizing components within the delivery vehicles that enhance the circulation lifetime of the carriers. In another aspect, the invention is directed to a method to deliver a therapeutic agent and a drug resistance modulator to a desired target by administering the compositions of the invention.

The invention is also directed to a method to deliver a therapeutically effective amount of a drug resistance modulator/therapeutic agent combination by administering a drug resistance modulator stably associated with a first delivery vehicle and a therapeutic agent stably associated with a second delivery vehicle. The first and second delivery vehicles may be contained in separate vials, the contents of the vials being administered to a patient simultaneously or sequentially. In one embodiment, the ratio of the drug resistance modulator and the therapeutic agent is non-antagonistic.

In another aspect, the invention provides a delivery vehicle composition comprising two or more drug resistance modulators in combination. Administration of such a composition allows for the delivery of multiple drug resistance modulators directed against multiple mechanisms that lead to drug resistance. The delivery vehicle composition may further comprise one or more therapeutic agents that are free or stably associated with a delivery vehicle. This invention is also directed to administering delivery vehicle compositions comprising two or more drug resistance modulators in combination.

In another aspect, the invention is directed to a method to deliver a therapeutically effective amount of two or more drug resistance modulators in combination, each drug resistance modulator being stably associated with a separate delivery vehicle. The delivery vehicles may be contained in separate vials, the contents of the vials being administered to a patient simultaneously or sequentially. In one embodiment, the ratio of the drug resistance modulators is non-antagonistic.

If the drug resistance modulators and/or therapeutic agents are not co-encapsulated, but are stably associated with separate delivery vehicles, the pharmacokinetics of the delivery vehicles should be coordinated. By “coordinated pharmacokinetics” is meant that the delivery vehicles behave in such a manner as so as to deliver the same ratio of active components to a target or tissue as was administered to the subject.

### BRIEF DESCRIPTION OF THE DRAWINGS

FIG. 1 is a diagram outlining an embodiment of the invention for determining an appropriate ratio of therapeutic agents to include in formulations.

### MODES OF CARRYING OUT THE INVENTION

In one aspect, the method of the invention involves stably associating a therapeutic agent and a drug resistance modulator in a delivery vehicle composition designed to treat drug resistance. This may be achieved by preparing the delivery vehicles to be of a size between 50-300 nm, more preferably 50-200 nm. As well, the delivery vehicles may contain lipid or non-lipid components to enhance blood stability. The therapeutic agent and the drug resistance modulator in combination should exhibit a biologic effect to a drug resistant target in vitro. Standard in vitro assays may be performed in order to determine whether drug resistance can be overcome by a combination of a therapeutic agent and a particular drug resistance modulator.

In another aspect, the invention involves stably associating two or more drug resistance modulators in a delivery vehicle composition in conjunction with one or more therapeutic agent. The therapeutic agent may be free or may be stably associated with delivery vehicles.

While it is preferred to co-encapsulate the therapeutic agents and the drug resistance modulator or the two or more drug resistance modulators in a single delivery vehicle, this is not necessary. Since particulate carriers can share similar pharmacokinetics, the substances experience coordinated delivery from the formulation even if encapsulated separately.

Delivery Vehicles

Delivery vehicles may include lipid carriers such as liposomes, lipid micelles, lipoprotein micelles, lipid-stabilized emulsions and polymer-lipid hybrid systems. Polymer nanoparticles, block copolymer micelles, cyclodextrins and derivatized single chain polymers may also be used.

Suitable lipid carriers for use in this invention are liposomes. Liposomes can be prepared as described in Liposomes: Rational Desig (A. S. Janoff, ed., Marcel Dekker, Inc., New York, N.Y.), or by additional techniques known to those knowledgeable in the art. Suitable liposomes for use in this invention include large unilamellar vesicles (LUVs), multilamellar vesicles (MLVs) and interdigitating fusion liposomes provided they are within a size range of 50 to 300 nm.

It should be readily apparent to those knowledgeable in the art that a number of lipid combinations could be employed to generate liposomes. Liposomes can be prepared by the incorporation of stabilizing lipids that specifically increase the blood residence time of the carrier. Examples of such lipids include phosphatidylglycerol (PG), phosphatidylinositol (PI), cholesterol (Chol) and hydrophilic polymer-lipid conjugates. Polymer-lipid conjugates such as PEG-lipid conjugates may be employed to shield a Liposome surface containing reactive components from immune recognition. The inclusion of a surface stabilizing polymer such as PEG can be used to stabilize liposomes containing lipids such as phosphatidylserine or antibodies. The incorporation of such components in liposomal compositions in the absence of surface stabilizing agents such as PEG results in the rapid recognition and clearance of the liposomes from the circulation. Thus, liposomes that contain a surface moiety, such as an antibody, that is not protected from interaction with blood components are not preferred for use in the invention. Optionally, liposomes of this invention are free of or contain low levels of such reactive components. An example of such a formulation is one containing a neutral lipid component such as phosphatidylcholine. Embodiments of this invention may make use of low cholesterol-containing liposomes (less than 30 mole % cholesterol in relation to the total lipid) containing PG or PI to prevent aggregation thereby increasing the blood residence time of the carrier. Preferably, the liposomes are free of cardiolipin.

Various techniques may be utilized to prepare stable liposomes of a suitable size. A particularly preferred method is the extrusion technique used to generate large unilamellar vesicles (LUVs). This method involves first combining lipids in chloroform to give a desired mole ratio. The resulting mixture is dried under a stream of nitrogen gas and placed in a vacuum pump until the solvent is substantially removed. The samples are then hydrated in an appropriate aqueous solution of a desired compound to be encapsulated. The mixture is then passed through an extrusion apparatus (e.g. apparatus by Northern Lipids, Vancouver, Canada) to obtain liposomes of a defined size. Optionally, sonication may be employed to produce small unilamellar vesicles (SUVs) of a size range between 20 and 50 nm. Liposomes prepared by sonication are not preferred for use in the invention.

The degree of saturation and the length of the acyl chains of lipid components making up a liposome may be adjusted to allow for optimal drug retention. The selection of lipids for incorporation into the liposome may be based on the attainment of a transition temperature that is above body temperature. Examples of lipids that will impart to the liposome a transition temperature above 37° C. include those in which the acyl chains are saturated and contain greater than 16 carbon atoms. An example of a particularly suitable lipid is DSPC.

Various methods may also be utilized to encapsulate active agents in liposomes. Examples of suitable loading techniques include conventional passive and active entrapment methods. Active methods of encapsulation include the pH gradient loading technique described in U.S. Pat. Nos. 5,616,341, 5,736,155 and 5,785,987. A preferred method of pH gradient loading is the citrate-base loading method utilizing citrate as the internal buffer at a pH of 4.0 and a neutral exterior buffer. Other methods employed to establish and maintain a pH gradient across a liposome involve the use of an ionophore that can insert into the liposome membrane and transport ions across membranes in exchange for protons (see U.S. Pat. No. 5,837,282). A recent technique utilizing transition metals to drive the uptake of drugs into liposomes via complexation in the absence of an ionophore may also be used. This technique relies on the formation of a drug-metal complex rather than the establishment of a pH gradient to drive uptake of drug.

Micelles are self-assembling particles composed of amphipathic lipids or polymeric components that are utilized for the delivery of sparingly soluble agents present in the hydrophobic core. Various means for the preparation of micellar delivery vehicles are available and may be carried out with ease by one skilled in the art. For instance, lipid micelles may be prepared as described in Perkins, et al., Int. J. Pharm. (2000) 200(1):27-39 (incorporated herein by reference). Lipoprotein micelles can be prepared from natural or artificial lipoproteins including low and high-density lipoproteins and chylomicrons. Lipid-stabilized emulsions are micelles prepared such that they comprise an oil filled core stabilized by an emulsifying component such as a monolayer or bilayer of lipids. The core may comprise fatty acid esters such as triacylglycerol (corn oil). The monolayer or bilayer may comprise a hydrophilic polymer lipid conjugate such as DSPE-PEG. These delivery vehicles may be prepared by homogenization of the oil in the presence of the polymer lipid conjugate. Agents that are incorporated into lipid-stabilized emulsions are generally poorly water-soluble. Synthetic polymer analogues that display properties similar to lipoproteins such as micelles of stearic acid esters or poly(ethylene oxide) block-poly(hydroxyethyl-L-aspartamide) and poly(ethylene oxide)-block-poly(hydroxyhexyl-L-aspartamide) may also be used in the practice of this invention (Lavasanifar, et al., J. Biomed. Mater. Res. (2000) 52:831-835).

Cyclodextrins comprise cavity-forming, water-soluble, oligosaccharides that can accommodate water-insoluble drugs in their cavities. Agents can be encapsulated into cyclodextrins using procedures known to those skilled in the art. For example, see Atwood, et al., Eds., “Inclusion Compounds,” Vols. 2 & 3, Academic Press, NY (1984); Bender, et al., “Cyclodextrin Chemistry,” Springer-Verlag, Berlin (1978); Szeitli, et al., “Cyclodextrins and Their Inclusion Complexes,” Akademiai Kiado, Budapest, Hungary (1982) and WO 00/40962.

Nanoparticles and microparticles may comprise a concentrated core of drug that is surrounded by a polymeric shell (nanocapsules) or as a solid or a liquid dispersed throughout a polymer matrix (nanospheres). General methods of preparing nanoparticles and microparticles are described by Soppimath, et al. (J. Control Release (2001) 70(1-2):1-20) the reference of which is incorporated herein. Nanoparticles in the size range of 50 to 200 nm are preferred for use in the invention. Other polymeric delivery vehicles that may be used include block copolymer micelles that comprise a drug containing a hydrophobic core surrounded by a hydrophilic shell; they are generally utilized as carriers for hydrophobic drugs and can be prepared as found in Allen, et al., Colloids and Surfaces B: Biointerfaces (1999) November 16(1-4):3-27. Polymer-lipid hybrid systems consist of a polymer nanoparticle surrounded by a lipid monolayer. The polymer particle serves as a cargo space for the incorporation of hydrophobic drugs while the lipid monolayer provides a stabilizing interference between the hydrophobic core and the external aqueous environment. Polymers such as polycaprolactone and poly(d,l-lactide) may be used while the lipid monolayer is typically composed of a mixture of lipid. Suitable methods of preparation are similar to those referenced above for polymer nanoparticles. Derivatized single chain polymers are polymers adapted for covalent linkage of a biologically active agent to form a polymer-drug conjugate. Numerous polymers have been proposed for synthesis of polymer-drug conjugates including polyaminoacids, polysaccharides such as dextrin or dextran, and synthetic polymers such as N-(2-hydroxypropyl)methacrylamide (HPMA) copolymer. Suitable methods of preparation are detailed in Veronese and Morpurgo, IL Farmaco (1999) 54(8):497-516 and are incorporated by reference herein.

Drug Resistance Modulators

The term “drug resistance modulator” refers to an agent that sensitizes a drug resistant target to the effects of one or more therapeutic agents. A drug resistance modulator may exhibit therapeutic activity on its own or may be inactive and thus act to potentiate the effect of a therapeutic agent. Approaches to sensitize drug resistance may involve reversing a mechanism that led to the development of the resistance. For the treatment of bacterial infections, examples of cellular functions that may be modulated include drug efflux mechanisms and de-activation of the anti-bacterial agent. In the case of cancer, mechanism-based treatments may involve treating dysregulated cellular pathways that lead to uncontrolled cell division, interfering with drug efflux mechanisms such as the functioning of the p-glycoprotein pump or by modulating mechanisms that inactivate drug. Since many chemotherapeutic agents ultimately elicit their effects via apoptosis, alterations at the level of apoptosis control provides a mechanism by which drug resistance may occur. As used herein, the term “apoptosis inducing agent” refers to agents that promote programmed cell death. Non-limiting examples of agents that induce apoptosis are pro-survival regulators, lipid mediators of apoptosis, cell cycle control inhibitors and signalling proteins such as GTPases. Examples of agents that fall within each class are given below.

Pro-survival regulators include members of the Bcl-2 family of proteins, which includes anti-apoptotic Bcl-2, BCl-x_{L} and Mcl-1 and pro-apoptotic Bax and Bak. The relative ratio of these proteins determine the sensitivity or resistance of cells to various apoptotic stimuli. Given that anti-apoptotic Bcl-2 family member proteins are expressed in many types of cancer that are drug resistant, the use of therapies directed against these targets may be employed in this invention. Inhibition of the activity of Bcl-2 family members may include the use of antisense molecules such as Genasense™, a Bcl-2 anti-sense molecule. Examples of drug combinations that may be used include Bcl-2 antisense in combination with low dose cyclophosphamide, paxlitaxel or dacarbazine. As well, Bcl-xL anti-sense may be used in combination with paclitaxel or dexamethasone.

Further examples of pro-survival regulators include Inhibitor of Apoptosis Proteins (IAPs), which are a family of apoptosis suppressor proteins that are believed to act as caspase inhibitors. Caspases contribute to many of the biochemical and morphological hallmarks of apoptosis. Since the binding of IAPs to caspases blocks caspase function, inhibitors of caspases may be used in this invention to induce apoptosis. A suitable method for down-regulating IAP family members is anti-sense therapy although other means of inhibition may be employed. Examples of antineoplastic agents that can be used in combination with IAP inhibitors include Taxol, cisplatin and etoposide. An example of an IAP family protein that may be downregulated to induce apoptosis is survivin.

Increasing the intracellular levels of bioactive lipids that induce apoptosis can also be employed to treat drug resistance. This may be carried out by the exogenous addition of a lipid that is known to induce an anti-cancer effect, or by indirectly modulating the activity of a mechanism that leads to increased intracellular levels of the bioactive lipid. Non-limiting examples of bioactive lipids that induce apoptosis include, sphingolipids such as ceramide and sphingosine, sphingosine analogs such as safingol and ether lipids such as edelfosine, edolfosine (ELL-12), ilmofosine and miltefosine.

Various agents known to those of skill in the art may be utilized to increase intracellular levels of bioactive lipids. An example is a glucosylceramide synthase inhibitor to increase intracellular levels of ceramide. Pharmacologic suppression of acid ceramidase by N-oleoylethanolamine (NOE) can be used to restore ceramide accumulation. Also sphingolipids that are sphingosine and ceramide derivatives can be generated with hydroxyl-replacement groups that block the bioconversion of ceramide to sphingolipids such as sphingomyelin, ceramide-1-phosphate, sphingosine, sphingosine-1-phosphate and glucosylceramide and thus result in enhanced intracellular ceramide content. Many such derivatives are detailed in Pei, et al. WO 95/21175 and U.S. Pat. No. 5,681,589, the contents of which are incorporated herein by reference. Studies have indicated that this is possible without inhibiting the signaling properties of the ceramide molecule. Increases in intracellular sphingosine, either by exogenous sphingosine or by treatment with the sphingosine kinase inhibitor dimethylsphingosine, can also be used to induce apoptosis.

Agents that affect the cell cycle in such a manner as to decrease uncontrolled cell proliferation may be used in the invention. Many human cancers are associated with genetic changes in cell cycle control pathways, which result in dysregulated cell proliferation. An example of a means of modulating the cell cycle involves inhibition of cyclin dependent kinase. Numerous pharmacologic inhibitors of Cdks have been investigated in an attempt to induce apoptosis and enhance the cytotoxicity of co-administered therapeutics. Non-limiting examples of Cdk inhibitors that may be used in this invention include 7-hydroxystaurosporine (UCN-01) and flavopiridol.

Signalling proteins also play a role in proliferative signals and thus the activity of these proteins may be regulated to decrease such proliferative signals. An example of a well-known signalling family of proteins that may be modulated to increase drug sensitivity are the GTPases, such as Rho and Ras. Various agents known to those of skill in the art may be used to modulate the cellular activity of signalling proteins. Examples of suitable agents include those that inhibit the activity of Ras by blocking farnesylation of the protein, such as farnesyl protein transferase enzymes. Three such inhibitors include BMS-214662, SCH66336 (Sarasar) and R115777 (Zarnestra™).

In addition to targeting mechanisms that regulate apoptosis, a further approach to sensitize cells to drug resistance involves inhibiting drug efflux systems. Membrane-bound drug pumps have been known to act singularly or in concert with other resistance mechanisms resulting in the clinical failure of anti-cancer agents. Increased efflux of drug from tumor cells may be the result of a drug pump such as ATP-binding cassette (ABC) transporters. Drug resistance conferred by these pumps is associated with an ATP-dependent reduced cellular accumulation of drug. Examples of compounds that may be used to inhibit the ability of drug transport proteins to efflux therapeutic agents from cells are given in U.S. Pat. No. 6,248,752, the contents of which are incorporated herein by reference.

P-glycoprotein is the most widely characterized ABC transporter that has been implicated in the development of a drug resistant phenotype. Since P-gp has been known to play a key role in the development of drug resistance, various approaches to inhibit the activity of this pump have been investigated. Non-limiting examples of agents that may be used to inhibit the activity of the P-gp drug pump in the practice of this invention include verapamil (Tsuruo et al., Cancer Res 1981 41:1967-1972), staurosporine (Sato et al., Biochem Biophys Res Commun (1990) 173:1252-1257), MS073, FK-506, cyclosporin A and its derivative PSC-833 (see Tsuruo in The Mechanism and New Approach on Drug Resistance of Cancer Cells. Amsterdam Elsevier Science Publishers, 1993; 81-91), indoloquinoxaline compounds such as PGP-4008 (Smith, et al, Oncol. Res. 12(5):219-29), hydrophobic peptide chemosensitizers such as Reversin 121 and 205 (Sharom, et al., Biochem. Pharm 58:571-586), LY-335979 which is a cyclopropyldibenzosuberane being developed by Eli Lily (Dantzig et al., Cancer Res. (1996) 56:4171-4179), XR9576 which is a novel anthranilic acid derivative (Mistry, et al., Cancer Res. (2001) 61:749-758), OC144-093 which is a novel diarylimidazole (Newman, et al., Cancer Res. (2000) 60:2964-2972), GF120918 which is an acridonecarboxamide derivative (Hyafil et al., Cancer Res (1993) 53:4595-4602) and VX710 which is a bispecific inhibitor of both PGP and MRP (Germann, et al., AntiCancer Drugs (1997) 8:125-140. Tamoxifen has also been shown to reverse P-170 glycoprotein-induced MDR in human and murine leukaemic cells (Berman et al., Blood (1991) 77:818-825). Antibodies directed against the p-glycoprotein pump may also be used to inhibit its function. Examples of such antibodies include MRK16, which has been shown to modulate vincristine and actinomycin D transport in resistant cells, (Tsuruo et al. Jpn J Cancer Res. (1989) 80:627-631) and MRK17 (Hamad and Tsuruo T. Proc Natl Acad Sci (1986) 83:7785-7789). Preferably, antibodies used in this invention are chimeric or humanized.

It has also been shown that P-gp is expressed in several subclasses of lymphocytes, including CD4 cells (Huisman et al, AIDS (2000) 14:237-242; Prechtl et al., J Immunol (2000) 164:754-761) and the accumulation of protease inhibitors were reduced by P-gp. Thus the inhibition of this transporter is also of relevance in the treatment of anti-HIV drug resistance.

Several other ABC transporter proteins have been implicated in the development of a drug resistant phenotype. These include the MDR-related protein (MRP) and the transporter of antigenic peptides (TAP). Non-limiting examples of modulators of MRP transport that may be used in the practice of the invention are indomethacin analogs such as 1-Benzoyl-5-methoxy-2-methylindole-3-acetic acid, 1-(4-Fluorobenzyl)-5-methoxy-2-metylindole-3-acetic acid and 1-(4-Chlorobenzyl)-5-methoxy-2-methylindole-3-acetic acid (Maguire, A. R., Bioorg. Med. Chem (2001) 9:745-762). MRP has also been implicated in the resistance of HIV drugs by extruding HIV protease inhibitors from the cell. Recently, it has been found that the protease inhibitor, Norvir, inhibits the efflux pump activity of MRP-1 in vitro, suggesting avenues for improved anti-HIV therapy (Olson et al., AIDS 2002 16(13):1743-1747). Thus, the inhibition of ABC transporter to treat drug resistance in HIV is also within the scope of the invention.

Lung-resistance-related protein (LRP) is another transporter that may be modulated to combat drug resistance in accordance with this invention. The protein induces drug efflux by controlling the movement of substrates between the nucleus and the cytoplasm. Thus, this transport mechanism is distinct from that of the ABC transporters. LRP was initially cloned from a non-small cell lung carcinoma cell line, which was resistant to doxorubicin, vincristine, etoposide and gramicidin D.

Drug detoxification systems also play an important role in eliminating chemotherapeutic agents and thus modulation of such systems is within the scope of this invention. An example of a detoxification system is glutathione-S-transferase (GST), which is overexpressed in many drug resistant cell lines. This enzyme conjugates reduced glutathione (GSH) to organic molecules in order to produce polar molecules that are readily excretable. Many anti-cancer agents, including nitrogen mustards and cyclophosphamides are biotransformed by GST enzymes. Non-limiting examples of inhibitors of GST that may be used are ethacrynic acid (Shen et al., Oncol. Res. (1997) 9:295-302) and buthionine sulfoximine (Batist et al., Biochem. Pharmacol (1991) 41:631-635) which deplete cells of intracellular glutathione content. Levels of GSH may be decreased by decreasing the intracellular synthesis of the compound. Sadzuka et al., have recently shown that this can be achieved by inhibition of a pump (GLAST or GLT-1) that transports glutamate across the cellular membrane (Toxicology Letters (2001) 123:159-167) by the amino acid theanine. GSH is synthesized from glutamate and therefore decreases in this molecule lead to decreases in intracellular GSH levels.

As in cancer, bacteria that develop drug resistance to antibiotics commonly possess a drug pump to reduce intracellular concentrations of drug (see Van Bambeke et al., Biochemical Pharmacology (2000) 60:457-470). The pumps are variants of membrane pumps possessed by all bacteria to move lipophilic or amphipathic molecules in and out of the cells. Drug efflux pumps have been observed in several bacteria, including staphylococci, which become resistant to the erythromycin class of macrolide antibiotics. Efflux pump inhibitors may thus be used in this invention to treat antibiotic resistance or antibiotics can be designed that are less susceptible to recognition and transit by a pump.

Another means by which bacteria develop drug resistance is through removal or destruction of functional groups within the antibiotic molecule. Antibiotics containing a β-lactam ring are known to be deactivated due to hydrolysis by the bacterial enzyme β-lactamase. Thus, drug resistance modulators that interfere with the functioning of this enzyme can be used in the invention. The drug, Clavulanate, a natural product from a streptomycete, has been shown to be an inhibitor of β-lactamase. The use of Clavulanate increased the anti-bacterial activity of the β-lactam drug, amoxicillin (The Choice of antibacterial drugs. Med. Lett. (1999) 41:95-104). A further example of a combination of a β-lactam antibiotic and a lactamase inactivator is Timentin and Zocin (Walsh, C. W. Nature (2000) 406:775-781).

Drug resistance may be a multifaceted phenomenon with numerous interacting pathways controlling its development. Thus, this invention is not limited to modulating one mechanism that leads to resistance. A multifunctional approach to combat drug resistance in which multiple pathways are targeted may be utilized. Thus, two or more drug resistance modulators may be stably associated with a delivery vehicle composition.

Although examples of agents that interfere with a resistance mechanism to sensitize cells to a particular disease condition are given above, it should be noted that the mechanism of drug sensitization may be unknown in some cases. Thus, it should be apparent to those of skill in the art that this invention also encompasses the use of agents that modulate drug resistance according to processes that, as of the present date, have not been elucidated.

Examples of Therapeutic Agents

Any suitable therapeutic agent may be combined with a drug resistance modulator in the practice of the invention. A “therapeutic agent” is a compound that alone, or in combination with other compounds, has a desirable effect on a subject affected by an unwanted condition or disease. A drug resistance modulator, as described above, can be combined with an agent that a particular target previously developed resistance to. Co-administration of the drug resistance modulator with this agent results in sensitization of a disease condition to the agent. As well, combinations containing greater than two agents are also within the scope of the invention.

Certain therapeutic agents are favored for use in combination with a drug resistance modulator when the target disease or condition is cancer. Examples are:

“Signal transduction inhibitors” which interfere with or prevent signals that cause cancer cells to grow or divide;

“Cytotoxic agents”;

“Cell cycle inhibitors” or “cell cycle control inhibitors” which interfere with the progress of a cell through its normal cell cycle, the life span of a cell, from the mitosis that gives it origin to the events following mitosis that divides it into daughter cells;

“Checkpoint inhibitors” which interfere with the normal function of cell cycle checkpoints, e.g., the S/G2 checkpoint, G2/M checkpoint and G1/S checkpoint;

“Topoisomerase inhibitors”, such as camptothecins, which interfere with topoisomerase I or II activity, enzymes necessary for DNA replication and transcription;

“Receptor tyrosine kinase inhibitors” which interfere with the activity of growth factor receptors that possess tyrosine kinase activity;

“Apoptosis inducing agents” which promote programmed cell death;

“Antimetabolites,” such as Gemcitabine or Hydroxyurea, which closely resemble an essential metabolite and therefore interfere with physiological reactions involving it;

“Telomerase inhibitors” which interfere with the activity of a telomerase, an enzyme that extends telomere length and extends the lifetime of the cell and its replicative capacity;

“Cyclin-dependent kinase inhibitors” which interfere with cyclin-dependent kinases that control the major steps between different phases of the cell cycle through phosphorylation of cell proteins such as histones, cytoskeletal proteins, transcription factors, tumor suppresser genes and the like;

“DNA damaging agents” Examples include carboplatin, cisplatin, cyclophosphamide, doxorubicin, daunorubicin, epirubicin, mitomycin C and mitoxantrone;

“DNA repair inhibitors”;

“Anti-angiogenic agents” which interfere with the generation of new blood vessels or growth of existing blood vessels that occurs during tumor growth; and

“Mitochondrial poisons” which directly or indirectly disrupt mitochondrial respiratory chain function.

Preferred agents that may be used in combination include DNA damaging agents such as carboplatin, cisplatin, cyclophosphamide, daunorubicin, epirubicin, mitomycin C, mitoxantrone; DNA repair inhibitors including 5-fluorouracil (5-FU) or FUDR, gemcitabine and methotrexate; topoisomerase I inhibitors such as camptothecin, irinotecan and topotecan; S/G2 or G2/M checkpoint inhibitors such as bleomycin, docetaxel, doxorubicin, etoposide, paclitaxel, vinblastine, vincristine, vindesine and vinorelbine; G1/early-S checkpoint inhibitors; G2/M checkpoint inhibitors; receptor tyrosine kinase inhibitors such as genistein, trastuzumab, ZD1839; cytotoxic agents; apoptosis-inducing agents and cell cycle control inhibitors. In one embodiment, the therapeutic agent is not doxorubicin.

Additional anticancer agents that are typically associated with drug resistance include those described in Shabbits et al., Exert Rev. Anticancer Ther. (2001) 1(4):585-594, the contents of which are incorporated herein by reference.

In Vitro Determination of Drug Sensitization

Standard in vitro assays may be performed in order to determine whether a drug resistant target can be sensitized to a particular therapeutic agent by the addition of a drug resistance modulator. When used in the context of the in vitro determination of drug sensitization, a “drug resistant target” is meant to include cells in culture including cells harvested from live tissue and bacterial and viral preparations. Whether or not a drug resistance modulator can sensitize a particular drug resistant target in vitro may simply involve a comparison of the biologic effect of the therapeutic agent alone and then in combination with the drug resistance modulator. An improvement in the biologic effect of the combination will be observed if the drug resistance modulator acts to allow the therapeutic agent to exert a desired effect. This result may be due to the inhibition of one or more mechanisms that led to drug resistance as described above. This method is preferred when the drug resistance modulator does not possess therapeutic activity on its own. As well, the ability of a drug resistance modulator to induce uptake of a therapeutic agent in vitro may be evaluated by the measuring the accumulation of drug within a cell in the absence and the presence of the modulator.

Optionally, a mathematical analysis of the in vitro assay results may be carried out in order to determine whether the combination exhibits an antagonistic or a non-antagonistic effect. The non-antagonistic effect may be synergistic or additive as defined by the Chou-Talalay method (Chou-Talalay median-effect method based on an equation described in Chou, J. Theor. Biol. (1976) 39:253-76; and Chou, Mol. Pharmacol. (1974) 10:235-247)) or by other standard data analysis methods detailed below. A potentiating effect will be observed if the drug resistance modulator is not effective by itself, but increases the effect of the therapeutic agent.

Various in vitro assays may be carried out in order to determine whether the delivery vehicle compositions of the invention are able to sensitize a drug resistant target. The in vitro studies on cell cultures may be conducted with relevant cells. The choice of cells will depend on the intended therapeutic use of the agent. Only one relevant cell line or cell culture type need exhibit the required biologic effect in order to provide a basis for the compositions to come within the scope of the invention.

For example, in one preferred embodiment of the invention, the combination of agents is intended for anticancer therapy. Appropriate choices will then be made of the cells to be tested and the nature of the test. In particular, tumor cell lines are suitable subjects and measurement of cell death or cell stasis is an appropriate end point. Also, in vitro studies on individual patient biopsies or whole tumors may be conducted with “cell homogenates,” generated from the mechanical or chemical disruption of the diseased tissues into single cells. As will further be discussed below, other target cells and criteria other than cytotoxicity or cell stasis could be employed. Cell death or viability may be measured using a number of methods known in the art. The “MTT” assay (Mosmann, J. Immunol. Methods (1983) 65(1-2):55-63) is preferred.

For determinations involving antitumor agents, cell lines resistant to one or more drugs may be obtained from standard cell line repositories (NCI or ATCC for example), from academic institutions or other organizations including commercial sources. Preferred drug resistant cell lines would include one or more selected from cell lines identified by the Developmental Therapeutics Program of the NCI/NIH. An example of a suitable cell line for use in this invention is the MDA435/LCC6 MDR cell line. Cell lines may be also be generated by the transfection of DNA or RNA rather than obtaining them from commercial sources.

It is known that a particular biologic effect observed by a combination of agents may be dependent on the ratio of the combination. It is possible that the same combination of drugs may be antagonistic at some ratios, synergistic at others, and additive at still others. Thus, in one embodiment, in order to prepare the compositions of the invention, the desired ratio of the drug resistance modulator and the therapeutic agent contained in the delivery vehicles is first determined. Desirably, the ratio will be that wherein synergy, potentiation or additivity is exhibited by the combination over a range of concentrations. Such ratios can be determined in vitro using various mathematical models.

Determination of ratios of agents that display synergistic or additive combination effects over concentration ranges may be carried out using various algorithms, based on the types of experimental data described below. These methods include isobologram methods (Loewe, et al., Arzneim-Forsch (1953) 3:285-290; Steel, et al., Int. J. Radiol. Oncol. Biol. Phys. (1979) 5:27-55), the fractional product method (Webb, Enzyme and Metabolic Inhibitors (1963) Vol. 1, pp. 1-5. New York: Academic Press), the Monte Carlo simulation method, CombiTool, ComboStat and the Chou-Talalay median-effect method based on an equation described in Chou, J. Theor. Biol. (1976) 39:253-76; and Chou, Mol. Pharmacol. (1974) 10:235-247). Alternatives include surviving fraction (Zoli, et al., Int. J. Cancer (1999) 80:413-416), percentage response to granulocyte/macrophage-colony forming unit compared with controls (Pannacciulli, et al., Anticancer Res. (1999) 19:409-412) and others (Berenbaum, Pharmacol. Rev. (1989) 41:93-141; Greco, et al., Pharmacol Rev. (1995) 47:331-385).

The Chou-Talalay median-effect method is preferred. The analysis utilizes an equation wherein the dose that causes a particular effect, fₐ, is given by:
D=Dₘ[fₐ/(1−fₐ)]^{1/m}
in which D is the dose of the drug used, fₐ is the fraction of cells affected by that dose, Dₘ is the dose for median effect signifying the potency and m is a coefficient representing the shape of the dose-effect curve (m is 1 for first order reactions).

This equation can be manipulated to calculate a combination index (CI) on the basis of the multiple drug effect equation as described by Chou and Talalay, Adv. Enzyme Reg. (1984) 22:27-55; and by Chou, et al., in: Synergism and Antagonism in Chemotherapy, Chou and Rideout, eds., Academic Press: New York 1991:223-244. A computer program for this calculation (CalcuSyn) is found in Chou and Chou, Dose-effect analysis with microcomputers: quantitation of ED50, LD50, synergism, antagonism, low-dose risk, receptor ligand binding and enzyme kinetics (CalcuSyn Manual and Software; Cambridge: Biosoft 1987).

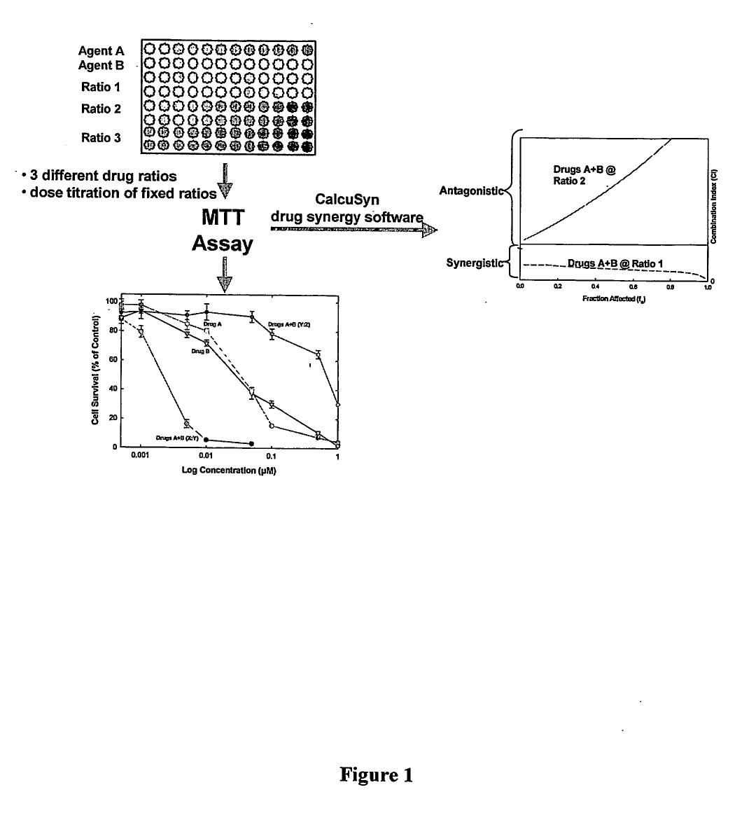

Preferably, the combination index (CI) is plotted as a function of the fraction of cells affected (fₐ) as shown in FIG. 1, which, as explained in the Example Section, is a surrogate parameter for concentration range. Preferred combinations of agents are those that display synergy, potentiation or additivity over a substantial range of fₐ values. Combinations of agents are selected that display synergy over at least 5% of the concentration range wherein greater than 1% of the cells are affected, i.e., an fₐ range greater than 0.01. Preferably, a larger portion of overall concentration exhibits a favorable CI; for example, 5% of an fₐ range of 0.2-0.8. More preferably 10% of this range exhibits a favorable CI. Even more preferably, 20% of the fₐ range, preferably over 50% and most preferably over at least 70% of the fₐ range of 0.2 to 0.8 are utilized in the compositions. Combinations that display synergy over a substantial range of fₐ values may be re-evaluated at a variety of agent ratios to define the optimal ratio to enhance the strength of the non-antagonistic interaction and increase the fₐ range over which synergy is observed.

While it would be desirable to have a non-antagonistic effect over the entire range of concentrations over which cells are affected, it has been observed that in many instances, the results are considerably more reliable in an fₐ range of 0.2-0.8. Thus, although the synergy exhibited by combinations of the invention is set forth to exist within the broad range of 0.01 or greater, it is preferable that the synergy be established in the fₐ range of 0.2-0.8.

The optimal combination ratio may be further used as a single pharmaceutical unit to determine synergistic, potentiating or additive interactions with a third agent. In addition, a three-agent combination may be used as a unit to determine non-antagonistic interactions with a fourth agent, and so on.

Combinations of drug resistance modulators and therapeutic agents may also be identified for their activity against microbial or viral infections or anti-inflammatory conditions that have developed drug resistance. As a first step in identifying antimicrobial agents, the minimum inhibitory concentration (MIC) for an agent can be determined by the classical microtitre broth dilution or agar dilution antimicrobial assays known to those skilled in the art. These assays are regulated by the National Committee of Laboratory Safety and Standards (NCLSS). The standard broth dilution assays are published in Amsterdam (1996) Susceptibility testing of Antimicrobials in liquid media in “Antibiotics in Laboratory Medicine”, Lorian, V. 4^{th} Edition, pages 52-111, Williams and Wilkins, Baltimore. The MIC is defined as the lowest concentration of an antibiotic that will inhibit the in vitro growth of an infectious organism. In the above-mentioned assays, the MIC can be determined by plating an inoculum of microbes in a small spot (at, for example, 10⁴ colony-forming units [CFU] per spot) on growth medium (for example, agar) having different concentrations of the drug. Alternatively, microbes can be inoculated into a suspension of growth media that contains different concentrations of the drug. In addition, the microbes may be either treated as above or may be resident as intracellular infections in a specific cell population (i.e., a macrophage). In the latter instance, mammalian cells grown in culture by standard methods are given intracellular microbial infections by brief exposure to a low concentration of microbes. After a period of time to allow the intracellular replication of the microbes, the cells and their intracellular microbes are treated with a drug in the same manner as described for cytotoxicity tests with mammalian cells. After an appropriate period of time sufficient for the drug to inhibit microbial growth when given at effective concentrations, the bacterial growth can be determined by a variety of means including: (i) determination of the absence or presence (and size, as appropriate) of the inoculum spot; (ii) plating and serial dilution of known volumes of the suspension of treated bacteria onto agar growth plates to allow calculation of the number of microbes that survived treatment; (iii) macroscopic (by eye) determination; (iv) time-kill curves in which microbes in the logarithmic phase of growth are suspended into a growth media containing a drug(s) and, at various times after inoculation, known volumes are removed and serial diluted onto growth agar for counting of the surviving microbes; (v) other spectroscopic, analytic, in vitro or in vivo methods known by those skilled in the art to allow the counting of viable microbes. The efficacy of a drug, or combinations of drugs to kill intracellular-resident infections are typically assessed after the host cells are lysed with detergents (such as 1% Triton X-100 plus 0.1% sodium dodecyl sulfate) to release the microbes, then the lysates are serial diluted onto agar growth plates for counting of the numbers of surviving microbes.

Extensive screening of agents or combinations of agents to sensitize viral preparations to antiviral agents can be performed by a number of in vitro assays, typically plaque reduction and cytopathic effects (CPE) inhibition assays. These assays are able to directly measure the extent to which an antiviral drug or drugs inhibits the effects of viral infection in tissue culture. The plaque reduction assay is preferred for virus and cell line combinations which produce a well-defined plaque. Michaelis, et al., demonstrated the use of plaque reduction assays combined with the Chou-Talalay method for determining non-antagonistic antiviral effects of aphidicolin and its derivatives on a number of viruses at various mole ratios (Michaelis, et al., Arzneimittelforschung (2002) 52(5):393-399). If a well-defined plaque is not producible by particular virus and cell line combinations, CPE inhibition assays are preferred. Additional methods for rapid and convenient identification of non-antagonistic combinations of antiviral agents include, but are not limited to, cell viability, virus yield and HIV acute or chronic infection assays. Cell viability is used to measure an antiviral agent's or combination of agent's ability to increase cell viability and can be achieved using quantitative assays such as the MTT assay previously described. Alternatively, the virus yield assay and the acute HIV infection assays evaluate an agent's ability to reduce virus yield allowing for direct measurements of antiviral activity. It will be apparent to those knowledgeable in the art that the aforementioned assays are suitable for screening antiviral drug combinations for synergistic, additive or antagonistic effects in vitro and are therefore included within the scope of the invention.

One skilled in the art will be able to define combinations of two or more agents selected for treatment of inflammatory disorders that have developed drug resistance by measuring, in vitro, suppression of proinflammatory cytokines such as IL-1, IL-18, COX-2, TNF or interferon-gamma. Other inflammatory signals include, but are not limited to, inhibition of prostaglandin E2 and thromboxane B2. In particular, endotoxin-mediated macrophage activation provides a suitable in vitro assay for measuring the anti-inflammatory effects of an added agent or combinations of agents and is commonly used in the art. In such an assay, macrophages grown in large quantities are activated by the addition of an endotoxin, such as lipopolysaccharide. Upon activation, macrophage secretion of cytokines such as IL-1 and TNF is measurable as well as activation of COX-2. Candidate anti-inflammatory drugs are added and evaluated based on their ability to suppress IL-1, TNF and COX-2. Titration with 1×10⁻⁷ M dexamethasone is typically used as a positive control. It will be apparent to those skilled in the art that assays involving macrophage activation are suitable for wide-spread screening of drug combinations and that suppression of IL-1, TNF and COX-2 are suitable endpoints for defining synergy. In addition to measuring inflammatory signals, investigators can consider the use of in vitro models that measure the effect of two or more agents on leukocyte functions. Functional tests can involve, but are not limited to, inhibition of degranulation, superoxide generation, and leukocyte migration.

Administering Delivery Vehicle Compositions

These delivery vehicle compositions may be used to treat a variety of diseases or conditions in warm-blooded animals and in avian species. In the treatment of cancer, vasculature is generally leakier than normal vasculature due to fenestrations or gaps in the endothelia. This allows the delivery vehicles of 300 m or less in diameter to penetrate the discontinuous endothelial cell layer and underlying basement membrane surrounding the vessels supplying blood to a tumor. Selective accumulation of the delivery vehicles into tumor sites following extravasation leads to enhanced delivery of the therapeutic agent and the drug resistance modulator.

As mentioned above, the delivery vehicle compositions of the present invention may be systemically administered to warm-blooded animals, including humans as well as to domestic avian species. For treatment of human ailments, a qualified physician will determine how the compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

Pharmaceutical compositions comprising delivery vehicles of the invention are prepared according to standard techniques and may comprise water, buffered water, 0.9% saline, 0.3% glycine, 5% dextrose and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable.

The concentration of delivery vehicles in the pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Preferably, the pharmaceutical compositions of the present invention are administered intravenously. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

### EXAMPLES

### Determination of Non-Antagonistic Ratios of a Drug Resistance Modulator and a Therapeutic Agent

In one embodiment, the method of the invention involves determining a ratio of a therapeutic drug and a drug resistance modulator which is non-antagonistic over a desired concentration range in vitro and supplying this non-antagonistic ratio in a manner that will ensure that the ratio is maintained at the site of desired activity. The non-antagonistic ratio is determined by applying standard analytical tools to the results obtained when at least one ratio of two or more therapeutic agents is tested in vitro over a range of concentrations against a particular drug resistant target such as cells in culture, cell homogenates, bacterial cells or viral preparations. By way of illustration, individual agents and various combinations thereof are tested for their biological effect on cells in culture or cell homogenates for example causing cell death or inhibiting cell growth, at various concentration levels. The concentration levels of the preset ratios are plotted against the percentage cell survival to obtain a correlation which can be manipulated by known and established mathematical techniques to calculate a “combination index” (CI). The mathematics are such that a CI of 1 (i.e., 0.9-1.1) describes an additive effect of the drugs; a CI>1 (i.e., >1.1) represents an antagonist effect; and a CI of <1 (i.e., <0.9) represents a synergistic or potentiating effect.

One general approach is shown in FIG. 1. As shown, agents A and B are tested individually and together at two different ratios for their ability to cause cell death or cell stasis as assessed by the MTT assay described below. Initially, correlations between the concentration of drugs A, B, and the two different combination ratios (Y:Z and X:Y) are plotted against cytotoxicity, calculated as a percentage based on the survival of untreated control cells. As expected, there is a dose-dependent effect on cell survival both for the individual drugs and for the combinations. Once this correlation has been established, the cell survival or fraction affected (fₐ) can be used as a surrogate for concentration in calculating the CI.

The results of the CI calculation are also shown in FIG. 1; this index is calculated as a function of the fraction of cells affected according to the procedure of Chou and Talalay, Advance Enz. Regul. (1985) 22:27-55. In this hypothetical situation, the first ratio (X:Y) of drugs A plus B is non-antagonistic at all concentrations while the combination in the second ratio (Y:Z) is antagonistic. Thus, it is possible to provide a ratio of drugs A plus B (ratio 1) which will be non-antagonistic regardless of concentration over a wide range. It is this ratio that is desirable to include in the compositions of the invention.

We claim:

1. A composition which comprises delivery vehicles, said delivery vehicles having a mean diameter in the range of 50-300 nm and having stably associated therewith at least one therapeutic agent and at least one drug resistance modulator, or at least two drug resistance modulators, wherein the therapeutic agent and the drug resistance modulator or the two drug resistance modulators in combination exhibit a biologic effect to a drug resistant target.

2. The composition of claim 1 wherein the biologic effect is non-antagonistic.

3. The composition of claim 1 wherein the therapeutic agent and the drug resistance modulator or the two drug resistance modulators are at a ratio that exhibits a non-antagonistic biologic effect to a drug resistant target.

4. The composition of claim 3 wherein the non-antagonistic biologic effect is a potentiating biologic effect.

5. The composition of claim 3 wherein the non-antagonistic biologic effect is a synergistic biologic effect.

6. The composition of claim 1 wherein the therapeutic agent is an antineoplastic agent.

7. The composition of claim 1 wherein the drug resistance modulator is a drug efflux pump inhibitor.

8. The composition of claim 7 wherein the drug efflux pump inhibitor is an inhibitor of p-glycoprotein.

9. The composition of claim 1 wherein the therapeutic agent and the drug resistance modulator in combination provide a therapeutic effect greater than either agent alone.

10. The composition of claim 1 wherein the therapeutic agent and the drug resistance modulator in combination provide a therapeutic effect to a tumor that is resistant to a first-line drug treatment.

11. The composition of claim 1 wherein said therapeutic agent is an anti-microbial agent.

12. The composition of claim 1 wherein therapeutic agent is an anti-viral agent.

13. The composition of claim 1 wherein the delivery vehicles have a circulation half-life of at least 2 hours.

14. The composition of claim 1 wherein the delivery vehicles are liposomes.

15. The composition of claim 14 wherein the liposomes are selected from the groups consisting of multilamellar vesicles (MLVs), large unilamellar vesicles (LUVs) and interdigitating fusion liposomes.

16. The composition of claim 14 wherein the liposomes comprise a stabilizing lipid.

17. The composition of claim 14 wherein the liposomes are not prepared by sonication.

18. The composition of claim 14 wherein the liposomes do not contain cardiolipin.

19. The composition of claim 1 wherein the therapeutic agent is not doxorubicin.

20. The composition of claim 1 wherein the delivery vehicles are nanoparticles.

21. A composition which comprises delivery vehicles, said delivery vehicles having stably associated therewith two or more drug resistance modulators.

22. The composition of claim 21 further comprising a therapeutic agent.

23. The composition of claim 22 wherein the therapeutic agent is not stably associated with a delivery vehicle.

24. The composition of claim 22 wherein the therapeutic agent is stably associated with a delivery vehicle.

25. The composition of claim 21 wherein the delivery vehicles have a mean diameter in the range of 50-300 nm.

26. The composition of claim 22 wherein the delivery vehicles have a mean diameter in the range of 50-300 nm.

27. A method to impart drug sensitivity and/or administer treatment to a patient which method comprises administering to said patient an effective amount of the composition of claim 1.

28. A method to impart drug sensitivity to a subject which method comprises administering to a subject in need of such drug sensitivity a composition comprising a first drug resistance modulators stably associated with a first delivery vehicle and a composition comprising a second resistance modulator stably associated with a second delivery vehicle wherein said first and second drug resistance modulators are administered at a ratio that is non-antagonistic and wherein the pharmacokinetics of the drug delivery vehicles in the first and second compositions are coordinated.

29. A method to administer a treatment to a subject in need of such treatment which method comprises administering to said subject a composition comprising a therapeutic agent stably associated with a first delivery vehicle and a drug resistance modulator stably associated with a second delivery vehicle at a ratio wherein said therapeutic agent and drug resistance modulator are non-antagonistic and wherein the pharmacokinetics of the first and second delivery vehicles are coordinated.

30. The composition of claim 2 wherein said non-antagonistic effect is exhibited over at least 5% of the concentration range such that 1%-99% of the cells are affected (fₐ=0.01-0.99) in an in vitro assay for cytotoxicity or cytostasis.

31. The composition of claim 30 wherein said non-antagonistic effect is exhibited over at least 5% of the concentration range such that 10-90% of the cells are affected (fₐ=0.1-0.9) in an in vitro assay for cytotoxicity or cytostasis.

32. The composition of claim 31 wherein said non-antagonistic effect is exhibited over at least 5% of the concentration range such that 20-80% of the cells are affected (fₐ=0.2-0.8) in an in vitro assay for cytotoxicity or cytostasis.

33. The composition of claim 32 wherein said synergistic effect is exhibited over at least 20% of the concentration range such that 20-80% of the cells are affected in an in vitro assay for cytotoxicity or cytostasis.

34. The method of claim 28 wherein said non-antagonistic effect is exhibited over at least 5% of the concentration range such that 1%-99% of the cells are affected (fₐ=0.01-0.99) in an in vitro assay for cytotoxicity or cytostasis.

35. The method of claim 34 wherein said non-antagonistic effect is exhibited over at least 5% of the concentration range such that 10-90% of the cells are affected (fₐ=0.1-0.9) in an in vitro assay for cytotoxicity or cytostasis.

36. The method of claim 35 wherein said non-antagonistic effect is exhibited over at least 5% of the concentration range such that 20-80% of the cells are affected (fₐ=0.2-0.8) in an in vitro assay for cytotoxicity or cytostasis.

37. The method of claim 36 wherein said synergistic effect is exhibited over at least 20% of the concentration range such that 20-80% of the cells are affected in an in vitro assay for cytotoxicity or cytostasis.

38. The method of claim 29 wherein said non-antagonistic effect is exhibited over at least 5% of the concentration range such that 1%-99% of the cells are affected (fₐ=0.01-0.99) in an in vitro assay for cytotoxicity or cytostasis.

39. The method of claim 38 wherein said non-antagonistic effect is exhibited over at least 5% of the concentration range such that 10-90% of the cells are affected (fₐ=0.1-0.9) in an in vitro assay for cytotoxicity or cytostasis.

40. The method of claim 39 wherein said non-antagonistic effect is exhibited over at least 5% of the concentration range such that 20-80% of the cells are affected (fₐ=0.2-0.8) in an in vitro assay for cytotoxicity or cytostasis.

41. The method of claim 40 wherein said synergistic effect is exhibited over at least 20% of the concentration range such that 20-80% of the cells are affected in an in vitro assay for cytotoxicity or cytostasis.
